(19) United States
(12) Patent Application Publication
(10) Pub. No.: US 2015/0190315 A1
(43) Pub. Date: Jul. 9, 2015

(54) CHEMICALLY TREATED CLOTH AND CLOTHING AND METHOD OF MANUFACTURE THEREOF

(71) Applicant: Michael A. Montano, Cincinnati, OH (US)

(72) Inventor: Michael A. Montano, Cincinnati, OH (US)

(21) Appl. No.: 14/590,809

(22) Filed: Jan. 6, 2015

Related U.S. Application Data
(60) Provisional application No. 61/924,956, filed on Jan. 8, 2014.

(51) Int. Cl. A61K 8/02; A61M 35/00; A61Q 19/00

(52) U.S. Cl. CPC A61K 8/0208; A61Q 19/00; A61M 35/00; A61K 2800/805

(57) ABSTRACT

A piece of cloth or clothing treated with a chemical application or combination of chemicals or ingredients which help in cleaning the skin and reducing acne. Other benefits may also be provided by the chemical application. One embodiment features a disposable hat insert which is treated to prevent breakouts along the forehead of the wearer of the hat. Chemicals or ingredients which would be appropriate for this application include, but are not limited to: Sodium Benzotriazolyl Butylphenol Sulfonate; Cocamidopropyl Betaine; C12-15 Alkyl Lactate; Witch Hazel (“Hammelis Virginiana”); Matricaria; Benzophenone; Aluminum Chloride; Salicylic Acid; Super Absorbent Polymers (e.g., Sodium Acrylate); Triclosan; Glycolic Acid; and Alpha Hydroxy Acid.

### CROSS-REFERENCE TO RELATED APPLICATION

This application claims priority in U.S. Provisional Patent Application No. 61/924,956, filed Jan. 8, 2014, which is incorporated herein by reference.

### BACKGROUND OF THE INVENTION

1. Field of the Invention

The present invention relates generally to chemically treated cloth and clothing, and more specifically to chemically treated pieces of cloth or clothing for reducing perspiration, cleaning skin, preventing odor, and dealing with other side effects of perspiration and a method of manufacture thereof.

2. Description of the Related Art

Clothing, especially clothing used while exercising or gym towels used to deal with perspiration, has the problem of absorbing sweat. Clothing and gym towels can often pick up germs or bacteria from the environment or may spread around germs or bacteria on a person's skin when they are perspiring and their pores are open and exposed. This can lead to break outs of the skin or other skin irritation.

Heretofore there has not been available a system or method for providing clothing and/or cloth for increasing skin care with the advantages and features of the present invention.

### BRIEF SUMMARY OF THE INVENTION

The present invention generally provides a system and method for applying a chemical application to an article of microfiber clothing; more specifically a chemical application applied to microfiber gym towels, microfiber wrist bands, headbands, t-shirts, or a disposable cap liner for inserting where the hat comes in contact with the forehead. These products aid in the prevention of acne breakouts due to sweat, oil, hair product residue, makeup and other chemicals that drip onto the face while working out. In addition, the chemically treated material will produce a cooling sensation and will kill bacteria that would normally be transferred back onto the face or skin when coming into contact with the cloth, such as a workout towel.

A variety of chemicals, used alone or in various combinations, may be applied directly to portions of a cloth item. The areas that are treated with the chemical(s) may be indicated using a different color material or other visual means. The chemicals or ingredients which would be appropriate for this application include, but are not limited to: Sodium Benzotriazolyl Butylphenol Sulfonate; Cocamidopropyl Betaine; C12-15 Alkyl Lactate; Witch Hazel (“Hammelis Virginiana”); Matricaria; Benzophenone; Aluminum Chloride; Salicylic Acid; Super Absorbent Polymers (e.g., Sodium Acrylate); Triclosan; Glycolic Acid; and Alpha Hydroxy Acid.

### BRIEF DESCRIPTION OF THE DRAWINGS

The drawings constitute a part of this specification and include exemplary embodiments of the present invention illustrating various objects and features thereof.

FIG. 1 is a top plan view of a baseball cap insert including a chemically treated portion.

FIG. 2 is a bottom plan view thereof.

FIG. 3 is a bottom plan view of a standard baseball cap including the cap insert thereof.

FIG. 4 is an isometric view of an alternative embodiment of the present invention featuring a wrist band.

FIG. 5 is a plan view of another alternative embodiment of the present invention featuring a headband.

FIG. 6 is a plan view of yet another alternative embodiment of the present invention featuring a headband.

FIG. 7 is a plan view of yet another alternative embodiment of the present invention featuring a headband.

FIG. 8 is an elevational view of yet another alternative embodiment of the present invention featuring a t-shirt.

FIG. 9 is an elevational view of yet another alternative embodiment of the present invention featuring a cloth towel.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

Cloth and clothing discussed herein can refer to any typical cloth or clothing material, such as cotton, microfiber, wool, spandex, or other suitable material capable of having a chemical additive introduced into the material.

The chemical application to the cloth or clothing material is intended to prevent breakouts on the skin while dealing with perspiration and other issues associated with perspiration. The chemicals or ingredients which would be appropriate for this application include, but are not limited to: Sodium Benzotriazolyl Butylphenol Sulfonate; Cocamidopropyl Betaine; C12-15 Alkyl Lactate; Witch Hazel (“Hammelis Virginiana”); Matricaria; Benzophenone; Aluminum Chloride; Salicylic Acid; Super Absorbent Polymers (e.g., Sodium Acrylate); Triclosan; Glycolic Acid; and Alpha Hydroxy Acid.

Sodium Benzotriazolyl Butylphenol Sulfonate may be used for its UV light stabilization properties which protects colors (e.g., dyes) and other light-sensitive ingredients introduced into the cloth or clothing item from photolytic and/or photo-oxidative degradation. It also improves the stability of formulations which are exposed to UV-A and UV-B light.

Cocamidopropyl Betaine may be used for its antiseptic properties, which makes it a suitable chemical for personal sanitary products.

C12-15 Alkyl Lactate may be used as a skin conditioning agent and/or an emollient.

Witch Hazel (“Hammelis Virginiana”) may be used for its strong anti-oxidant and astringent properties which is useful for fighting and preventing acne.

Matricaria may be used for its anti-inflammatory purposes. Matricaria is often used in ointments and lotions for these purposes, and could be incorporated into a chemical combination applied to a cloth or portion of cloth material.

Benzophenone prevents ultraviolet (UV) light from damaging scents and colors in products, similar to Sodium Benzotriazolyl Butylphenol. It can also be added to the plastic packaging as a UV blocker. This allows manufacturers to package the cloth in clear plastic and not limit packaging to opaque material.

Aluminum Chloride may be used for its perspiration-blocking properties.

Salicylic Acid may be used to aid in clearing pores and preventing acne.

Super Absorbent Polymers (e.g., Sodium Acrylate) may be used to absorb perspiration. These polymers are capable of absorbing 500-1,000 times their mass in water and provide excellent stability under heat and light.

Triclosan may be used to reduce or prevent bacterial contamination of the cloth or clothing.

Glycolic Acid may be used to help clear pores and reduce the formation of whiteheads and blackheads in the skin.

Alpha Hydroxy Acid may be used for its ability to moisturize and exfoliate the skin, producing an improved look and feel of the skin.

### II. Preferred Embodiment Treated Insert 2

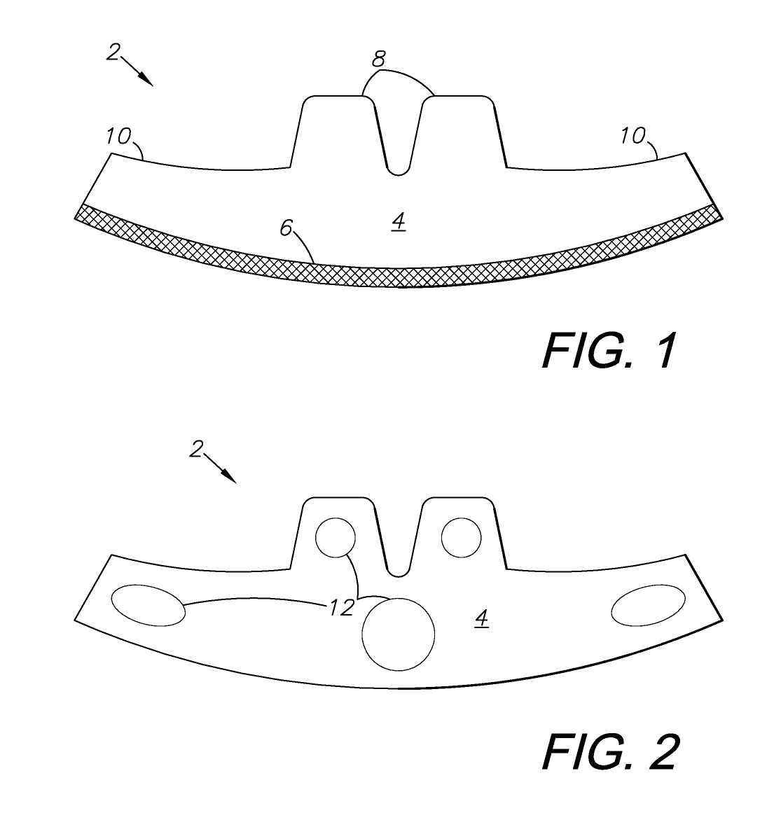

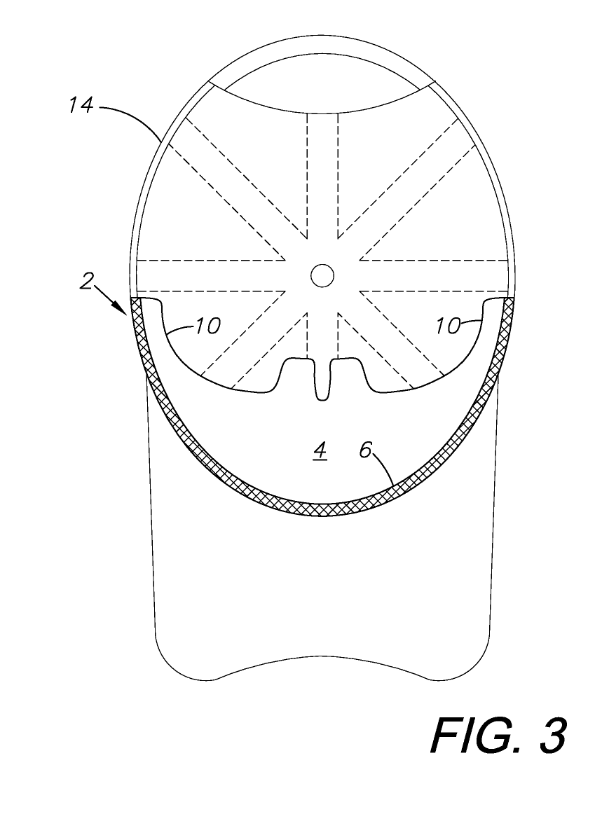

Referring to the figures in more detail, FIGS. 1-3 show a treated insert 2 for a baseball cap 14 as an example of a treated piece of clothing or cloth material. The insert 2 has an untreated main portion 4 with a pair of wings 10 for fitting along the brim of a baseball cap 14, a pair of tabs 8 for helping to secure the insert within the hat, a treated portion 6 which contains the chemical application, and several adhesive patches 12 for helping to secure the insert 2 within the hat 14.

The insert 2 is designed for use in a baseball cap 14, but could be adapted for any hat. The purpose of the insert is to allow the treated portion 6 of the insert 2 to make contact with the forehead, thereby preventing sweat and dirt from clogging pores and causing breakouts. The chemical application applied to the treat portion 6 may also include elements which produce a cooling sensation.

The insert is disposable and is meant to be worn whenever the hat is in use, particularly when the user is performing athletic activities or other work where the user is likely to perspire.

### III. Alternative Embodiment Treated Wristband 52

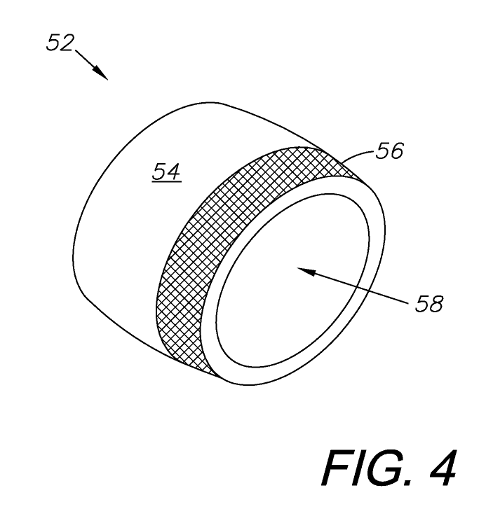

FIG. 4 shows a wristband 52 which has an untreated portion 54, a treated portion 56, and an opening 58 for a wearer's wrist to extend through the wristband 52. The treated portion could encompass up to the entire outer surface of the wristband 52. The wrist band can be used while exercising or working to wipe perspiration from the user's forehead. The treated portion 56 may be colored differently from the untreated portion 54, and may form a decorative stripe as shown in FIG. 4.

### IV. Alternative Embodiment Treated Headband 102

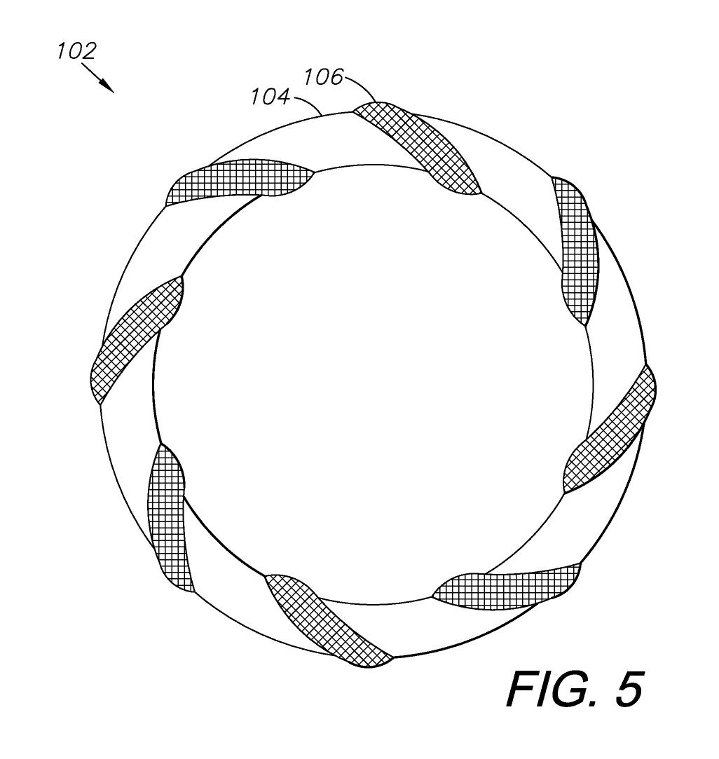

FIG. 5 shows a twisted headband 102 which has an untreated portion 104 twisted with a treated portion 106. The headband can be used while exercising or working to soak up perspiration from the user's forehead. The treated portion 106 may be colored differently from the untreated portion 104. Any metal-based or hard plastic-based connecting elements (not shown) could be tucked between the two portions to prevent those elements from scratching the skin.

### V. Alternative Embodiment Treated Headband 152

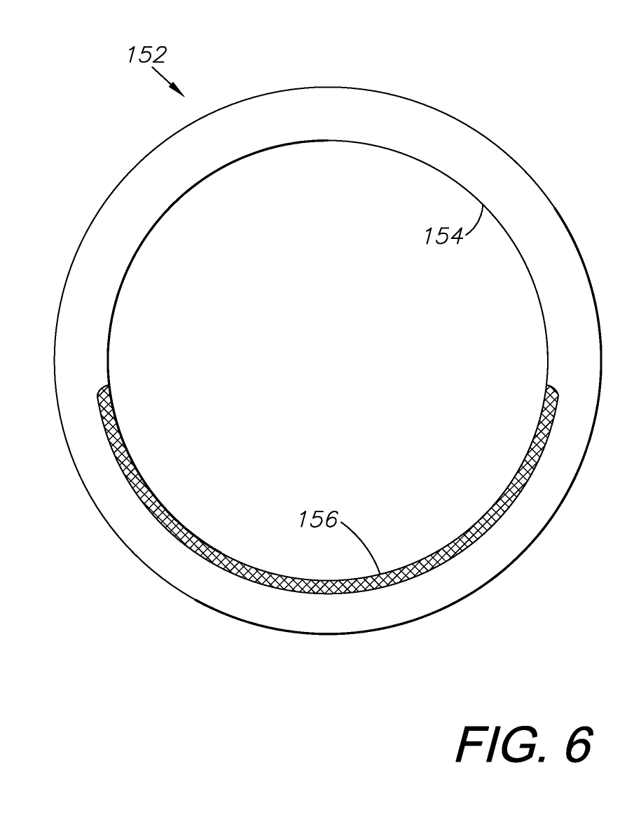

FIG. 6 shows a headband 152 with a treated portion 156 and an untreated portion 154. The treated portion 156 is intended to be placed against the wearer's forehead to prevent breakouts and other skin irritation.

### VI. Alternative Embodiment Treated Headband 202

FIG. 6 shows a headband 202 with a treated portion 206 and an untreated portion 204. The treated portion 206 is intended to be placed against the wearer's forehead to prevent breakouts and other skin irritation. This embodiment differs from the previous embodiment 152 whereas the treated portion 206 is located around the entirety of the inner ring of the headband 202 instead of being located only along a portion of the inner ring.

### VII. Alternative Embodiment T-Shirt 252

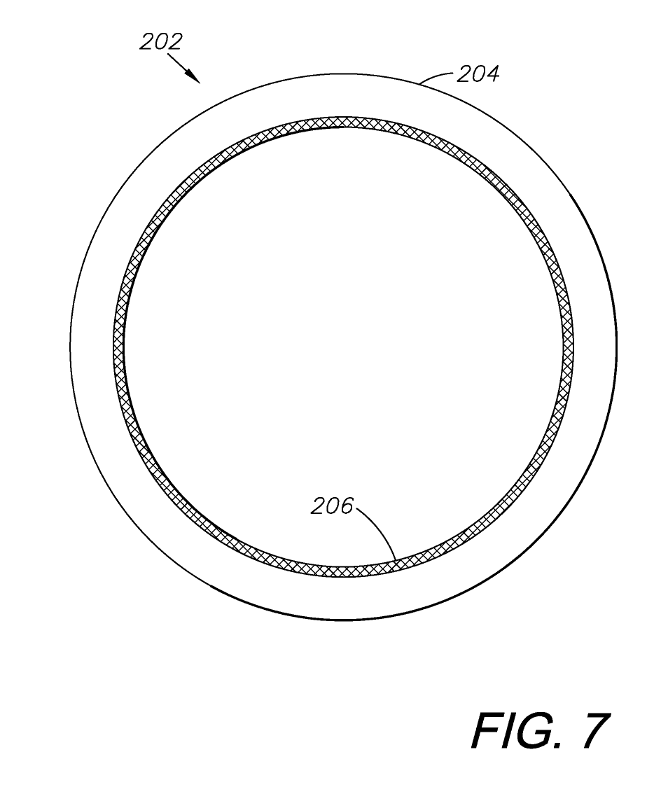

FIG. 7 shows a T-Shirt 252 with an untreated neck portion 254, a treated neck portion 256 and the rest of the garment 258. The inside of the neck portion is the chemically treated portion. This garment can be a T-Shirt as shown or any other suitable piece of apparel where portions of the apparel make frequent contact with the skin.

VIII. Alternative Embodiment Towel 302

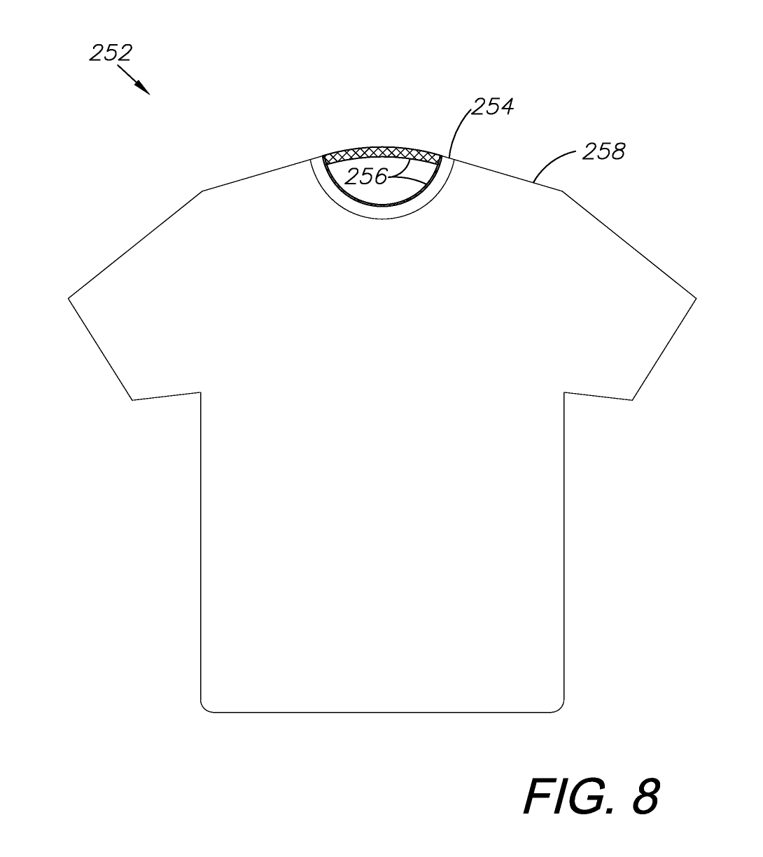

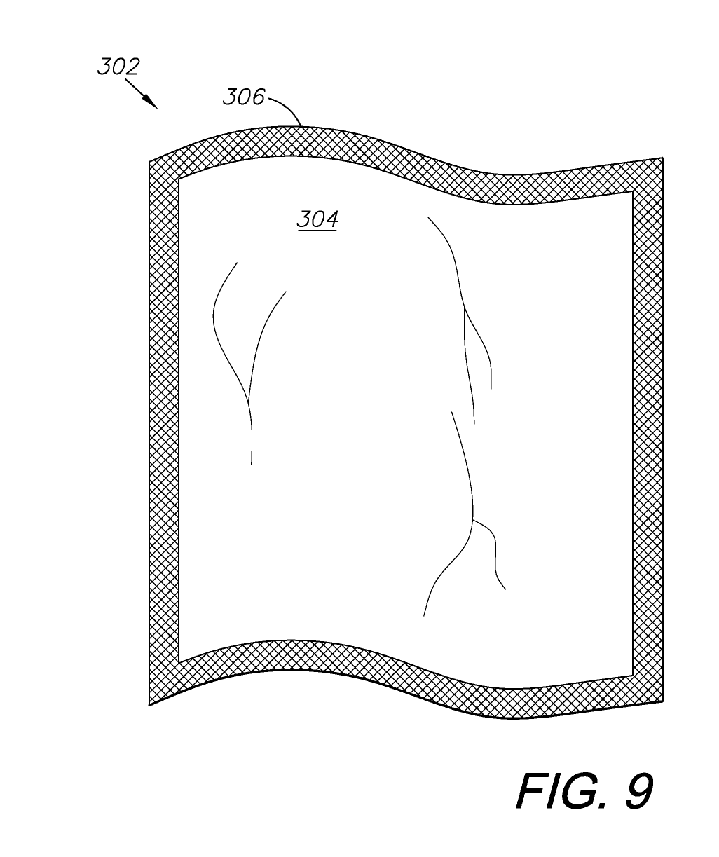

People often use towels while working out to remove perspiration from the face and body. This can result in transferring bacteria to the towel which can then be spread about the body, increasing the likelihood of forming acne on the skin. FIG. 8 shows a workout towel 302 featuring a treated face 304 indicated by a border 306 which indicates which face of the towel is treated with the chemical application. The opposing face (not shown) does not include a border indicating chemical application.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. A cloth-based apparatus comprising:
a body;
an untreated portion of said body;
a treated portion of said body located adjacent to said untreated portion, said treated portion treated with a combination of ingredients configured to improve skin health; and
wherein said treated portion comprises an indicator configured to indicate where the treated portion is located.

2. The apparatus of claim 1, wherein said combination of ingredients comprises one or more of the ingredients selected from the list comprising: Sodium Benzotriazolyl Butylphenol Sulfonate; Cocamidopropyl Betaine; C12-15 Alkyl Lactate; Witch Hazel; Matricaria; Benzophenone; Aluminum Chloride; Salicylic Acid; Super Absorbent Polymers; Sodium Acrylate; Triclosan; Glycolic Acid; and Alpha Hydroxy Acid.

3. The apparatus of claim 1, further comprising:
said body configured to be insert into a hat, said body comprising a pair of wings, an outer face, an inner face, a front edge, and a back edge comprising a pair of tabs;
said inner face including a plurality of adhesive points for adhering to an inner surface of said hat; and
said treated portion being located along said front edge on said outer face in proximity to the brim of said hat, wherein said treated portion is configured to be placed against a forehead.

4. The apparatus of claim 3, further comprising:
said wings configured to wrap around the inner surface of said hat; and
said tabs configured to conform to said inner surface of said hat.

5. The apparatus of claim 1, further comprising:
said body forming a stretchable band adapted for absorbing perspiration.

6. The apparatus of claim 5, wherein said body is configured to be placed about a wearer's wrist.

7. The apparatus of claim 5, wherein said body is configure to be placed about a wearer's head, including covering at least a portion of a forehead with said treated portion.

8. The apparatus of claim 7, wherein said treated portion comprises a second stretchable band configured to be intertwined with said body.

9. The apparatus of claim 5, further comprising said body including an inner surface and an outer surface.

10. The apparatus of claim 9, wherein said treated portion comprises less than half of said inner surface.

11. The apparatus of claim 9, wherein said treated portion comprises the entire inner surface.

12. The apparatus of claim 1, further comprising:
wherein said body comprises a shirt including a neck hole having an inner surface and an outer surface; and
wherein said treated portion comprises said inner surface of said body.

13. The apparatus of claim 1, further comprising:
wherein said body comprises a towel having a first side and a second side;
said first side comprising said treated portion; and
wherein said indicator comprises a border surrounding said first side.

14. A baseball cap insert comprising:
a body including a treated portion and an untreated portion;
an untreated portion of said body;
said body configured to be insert into a hat, said body comprising a pair of wings, an outer face, an inner face, a front edge, and a back edge comprising a pair of tabs;
said inner face including a plurality of adhesive points for adhering to an inner surface of said hat;
said treated portion being located along said front edge on said outer face in proximity to the brim of said hat, wherein said treated portion is configured to be placed against a forehead;
said wings configured to wrap around the inner surface of said hat;
said tabs configured to conform to said inner surface of said hat;
said treated portion treated with a combination of ingredients configured to improve skin health; and
an indicator in proximity to said treated portion configured to indicate where the treated portion is located.
